(19) United States
(12) Patent Application Publication
(10) Pub. No.: US 2023/0149118 A1
(43) Pub. Date: May 18, 2023

(54) DEVICES, SYSTEMS, AND METHODS FOR FLUID DELIVERY

(71) Applicant: Boston Scientific Scimed, Inc., Maple Grove, MN (US)

(72) Inventors: Patrick A. Haverkost, Corcoran, MN (US); Bhanu Prasanth Koppolu, Cary, NC (US); Joel N. Groff, Delano, MN (US); Kyle Alan Trepanier, Circle Pines, MN (US)

(73) Assignee: Boston Scientific Scimed, Inc., Maple Grove, MN (US)

(21) Appl. No.: 17/988,114

(22) Filed: Nov. 16, 2022

Related U.S. Application Data
(60) Provisional application No. 63/279,727, filed on Nov. 16, 2021.

(51) Int. Cl. A61B 90/00

(52) U.S. Cl. CPC A61B 90/39; A61B 2090/3987

(57) ABSTRACT

Fluid delivery devices, systems, and methods disclosed herein may include a cannula that may include a shaft comprising a proximal end, a distal end, and a lumen. A plurality of radial apertures may be disposed along a delivery portion of the shaft. Each radial aperture may be in fluid communication with the lumen. At least one echogenic marker may be disposed along the shaft within or adjacent to the delivery portion. A body may be disposed within the distal end of the shaft.

### CROSS REFERENCE TO RELATED APPLICATIONS

This application claims the benefit of and priority to U.S. Provisional Patent Application Ser. No. 63/279,727, filed on Nov. 16, 2021, the disclosure of which is incorporated herein by reference.

### FIELD

The present disclosure relates generally to the field of medical devices. In particular, the present disclosure relates to medical devices for delivering a fluid to a target location, such as a diseased tissue, e.g., a tumor or the like.

### BACKGROUND

Delivering fluids to treatment sites may include targeting delivery to particular tissues or a portion of a tissue, e.g., a tumor. It may be desirable for the fluid to only be delivered to the target site with minimal fluid engagement with ancillary tissue(s). For example, in oncology, the fluid may have destructive effects on tissue that would be undesirable among healthy tissue. Therefore, targeted percutaneous cannula delivery may be a desirable method of fluid delivery to the site to ensure substantially localized fluid deliver. However, such fluid delivery techniques may have complications. Although the fluid may be delivered to the site, it may be such a low viscosity that it flows and/or leeches outside of the target site upon delivery or over time after delivery. The fluid may have such a high viscosity that the dimensional parameters of the delivery cannula may not effectively deliver, deploy, disperse, or maintain flow of the fluid to the target site. The cannula and/or the target site may also be difficult for a medical professional to visualize and/or navigate for desirable fluid delivery.

Accordingly, a variety of advantageous medical outcomes addressing the above deficiencies may be realized by the devices, systems, and methods of the disclosure.

### SUMMARY

Medical devices, systems, and methods are described herein, e.g., for facilitating fluid delivery to a treatment site, such as a tumor. In one aspect, a cannula may comprise a shaft comprising a proximal end, a distal end, and a lumen. A plurality of apertures may be disposed along a delivery portion of the shaft. Each aperture may be in fluid communication with the lumen. Each aperture may extend radially from a central axis of the shaft. At least one echogenic marker may be disposed along the shaft within or adjacent to the delivery portion. A body may be disposed within the distal end of the shaft. The lumen may extend along an axial length of the shaft, and a body may be disposed within a distal end of the lumen, blocking the distal end of the lumen.

In the described and other aspects of the present disclosure, the body may comprises a pointed distal tip, a midportion having a diameter larger than the distal tip, and a proximal insertion portion having a diameter smaller than the midportion and dimensioned to fit within the distal end of the shaft. The diameter of the midportion may be greater than or equal to a diameter of the shaft. The cannula may comprise 1 to 100 apertures or more. The plurality of apertures may comprise at least one ringed pattern of apertures at a flexible portion along the shaft. The plurality of apertures may comprise at least one helical pattern of apertures along the shaft. The plurality of apertures may be circular in shape, oblong in shape, ellipsoidal in shape, rectangular in shape, or a combination thereof. The plurality of apertures may comprise at least one proximal aperture having a smaller width than a width of at least one distal aperture of the plurality of apertures. The plurality of apertures may comprise a gradual increase in width along a length of the shaft between a proximal end of the delivery portion and a distal end of the delivery portion. A width of at least one of the plurality of apertures may be about 0.254 mm±0.100 mm to about 0.381 mm±0.100 mm. An axial length of the delivery portion may range from about 1.5 cm to about 2.5 cm. The cannula may comprise one or more echogenic markers disposed adjacent a proximal end of the delivery portion and/or one or more echogenic markers disposed adjacent a distal end of the delivery portion. The cannula may comprise a plurality of echogenic markers in the form of rings disposed adjacent a proximal end of the delivery portion and/or a plurality of echogenic markers in the form of rings disposed adjacent a distal end of the delivery portion. An axial length of the at least one echogenic marker may range from about 2 mm to about 10 mm.

In one aspect, a cannula may comprise a shaft comprising a proximal end, a distal end, and a lumen therethrough. A plurality of apertures may be disposed along a delivery portion of the shaft. Each aperture may be in fluid communication with the lumen. Each aperture may extend radially from a central axis of the shaft. The plurality of apertures may be circular in shape, oblong in shape, ellipsoidal in shape, rectangular in shape, or a combination thereof. A distal marker portion may be longitudinally along the shaft distal to the delivery portion. A proximal marker portion may be along the shaft proximal to the delivery portion. A body may be disposed within the distal end of the shaft. The lumen may extend along an axial length of the shaft, and a body may be disposed within a distal end of the lumen, blocking the distal end of the lumen. The distal body may extend distally from the shaft to a gradually pointed tip.

In the described and other aspects of the present disclosure, each of the distal marker portion and the proximal marker portion may comprise a plurality of echogenic ringed grooves. The body may comprise a pointed distal tip, a midportion having a diameter larger than the distal tip, and a proximal insertion portion having a diameter smaller than the midportion and dimensioned to fit within the distal end of the shaft. A width of at least one of the plurality of apertures may be about 0.254 mm to about 0.381 mm.

In one aspect, a method of delivering a fluid disclosed herein may include inserting a cannula comprising a shaft, a lumen, and a plurality of apertures disposed along a delivery portion of the shaft into a patient. Each aperture may extend radially from a central axis of the shaft. The plurality of apertures may be circular in shape, oblong in shape, ellipsoidal in shape, rectangular in shape, or a combination thereof. Each aperture may be in fluid communication with the lumen. At least one echogenic marker may be disposed along the shaft within or adjacent to the delivery portion. At least one echogenic marker may be detected with ultrasound. The delivery portion may be positioned within a target tissue with the assistance of the at least one echogenic marker. The fluid may be delivered through the lumen and out of the plurality of apertures such that the fluid is delivered to the target tissue.

In the described and other aspects of the present disclosure, the fluid may comprise a viscosity of fluids such as solutions including water, saline and hydrogels that may vary based on composition, temperature, and concentration. The fluid may comprise a viscosity that increases as the fluid approaches a body temperature of a patient. The at least one echogenic marker may be positioned at a border of the target tissue. The fluid may be a gellable fluid. The fluid may be maintained at a temperature below about 37° C. at least until delivery. The delivery portion may be oriented along the target tissue by flexing the delivery portion. The fluid may be maintained within the target tissue for a treatment period of at least 24 hours or more.

### BRIEF DESCRIPTION OF THE DRAWINGS

Non-limiting examples of the present disclosure are described with reference to the accompanying figures, which are schematic and not intended to be drawn to scale. In the figures, each identical or nearly identical component illustrated is typically represented by a single numeral. For purposes of clarity, not every component is labeled in every figure, nor is every component in each embodiment of the disclosure shown where illustration is not necessary to allow those of skill in the art to understand the disclosure. In the figures:

FIG. 1 illustrates a perspective view of a cannula delivering a fluid to a target tissue, according to an embodiment of the disclosure.

FIG. 2A illustrates a perspective view of a shaft, according to an embodiment of the disclosure.

FIG. 2B illustrates a perspective view of a body, according to an embodiment of the disclosure.

FIG. 2C illustrates the body of FIG. 2B and a handle in an exploded perspective view with the shaft of FIG. 2A, according to an embodiment of the disclosure.

FIG. 2D illustrates a perspective view of a distal end of the assembled shaft and body of FIGS. 2A-2C, according to an embodiment of the disclosure.

FIG. 2E illustrates an alternative perspective view of a distal end of a shaft and body, according to an embodiment of the disclosure.

FIG. 3 illustrates a perspective view of an array of bodies, according to an embodiment of the disclosure.

FIG. 4 illustrates a perspective view of a fluid delivery system, according to an embodiment of the disclosure.

FIG. 5 illustrates a perspective view of a shaft, according to an embodiment of the disclosure.

FIG. 6 illustrates a perspective view of a shaft, according to an embodiment of the disclosure.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs. The detailed description should be read with reference to the drawings, which are not necessarily to scale, depict illustrative embodiments, and are not intended to limit the scope of the invention.

As used herein, “proximal end” refers to the end of a device that lies closest to the medical professional along the device when introducing, removing, or exchanging the device within a patient, and “distal end” refers to the end of a device or object that lies furthest from the medical professional along the device during implantation, positioning, or delivery.

As used in this disclosure and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g., 1 to 5 includes 1, 1.5, 2, 2.75, 3, 3.80, 4, and 5).

Although embodiments of the present disclosure are described with specific reference to cannulas, it should be appreciated that other medical devices may be used in a variety of medical procedures. For example, a cannula described herein may instead be a catheter, or the like, and may be percutaneous or non-percutaneous.

As used herein, “fluid” may comprise one or more of a liquid, semi-liquid, super critical fluid, gas, solution, particles, drug carriers, spheres, a combination thereof, or the like. A fluid herein may comprise a viscosity that increases as its temperature increases. For example, a fluid may have a lower viscosity at a first temperature and a higher viscosity at a second temperature. For an even more specific example, a fluid may be delivered at a lower temperature within a cannula and may be deployed at a higher temperature (e.g., at least 37° C.) with a higher viscosity. Once deployed, a lower viscosity fluid may undesirably flow through and among a target site and adjacent tissue compared to a higher viscosity fluid that may desirably not flow substantially away from the target site during and/or after deployment. In some embodiments, a fluid is delivered which is a reverse thermosensitive fluid. These fluids are liquids below body temperature and viscous gels at body temperature. In various embodiments, the fluid is provided external of the body at a temperature below body temperature. The fluid may be further chilled to prolong the time the gel stays in the liquid form upon introduction into the body, in some embodiments. For example, the introduction temperature may be is about 10° C. or more below the gelation temperature of the fluid.

Although embodiments of the present disclosure are described with specific reference to oncologic fluid delivery into a tumor, it should be appreciated that embodiments herein may deliver fluid in a variety of medical treatment sites such as tissues, organs, body lumens, ducts, vessels, fistulas, cysts, and spaces (e.g., the dermis, stomach, duodenum, jejunum, small intestine, gallbladder, kidneys, pancreas, biliary trees, pancreatic trees, bladder, ureter, abscesses, walled-off pancreatic necrosis, bile ducts, etc.). Devices herein may be inserted via different access points and approaches, e.g., percutaneously, endoscopically, laparoscopically or some combination thereof.

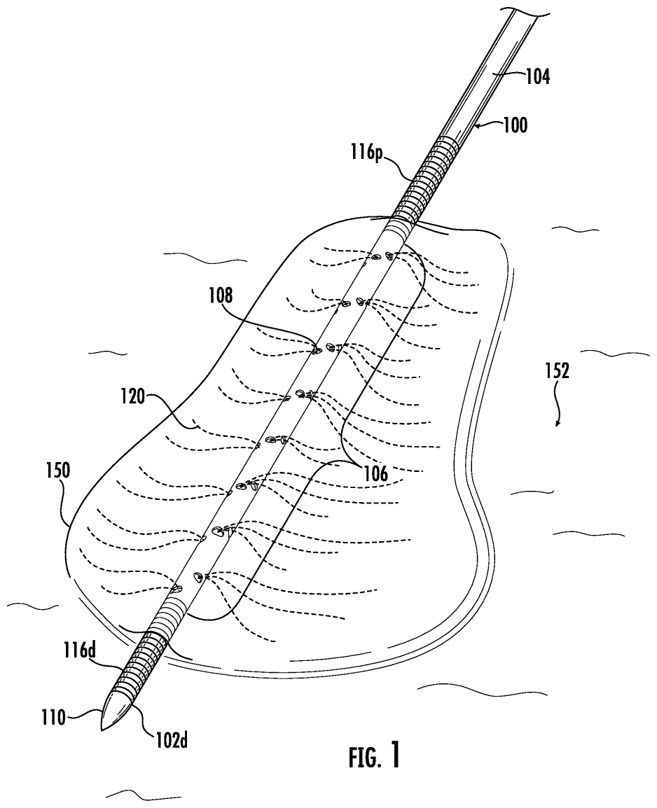

Referring to FIG. 1A, a perspective view of a cannula 100 delivering fluid 120 into a target tissue 150 is illustrated, according to an embodiment of the disclosure. The cannula 100 includes a shaft 102 having a proximal end (not shown), a distal end 102d, and a lumen 104. Radial apertures 108 are disposed along a delivery portion 106 of the shaft 102. The radial apertures 108 are disposed helically about the delivery portion 106 of the shaft 102 and each radial aperture 108 is in fluid communication with the lumen 104. The fluid 120 is being delivered through the lumen 104 and the fluid 120 is being deployed from the lumen 104 out of the apertures 108. A body 110 is disposed within the distal end 102d of the shaft 102. The body 110 includes a pointed tip that may assist the cannula 100 with entering one or more tissues such as the target tissue 150 and/or adjacent tissue 152. The body 110 occludes a distal end of the lumen 104 such that the fluid 120 egresses only through the apertures 108. There is a proximal echogenic marker 116p and distal echogenic marker 116d disposed along the shaft 102, each adjacent to the delivery portion 106. The echogenic markers 116p, 116d are placed across the border of the target tissue 150 such that the delivery portion 106 of the shaft 102 is located entirely within the target tissue 150. With this arrangement, the fluid 120 is delivered and deployed into the target tissue 150 while discouraging fluid from entering the adjacent tissue 152.

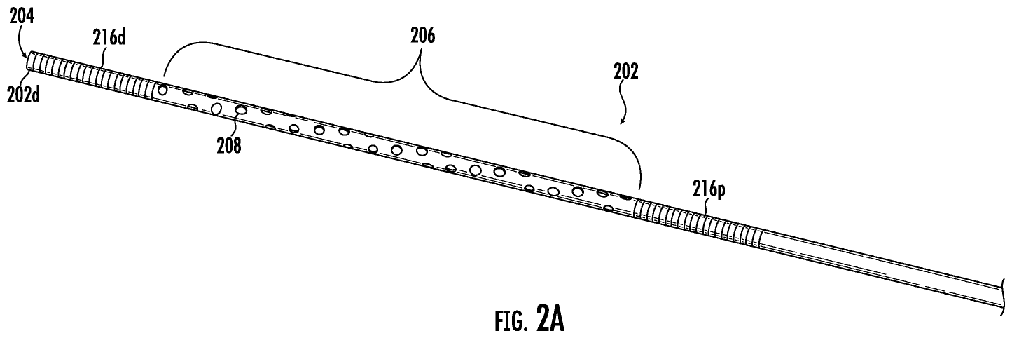

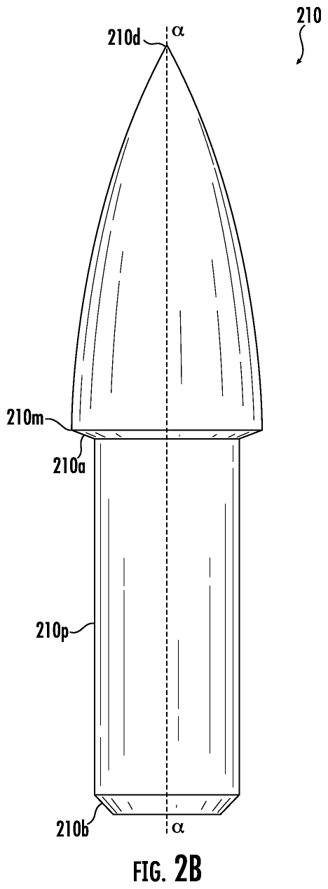

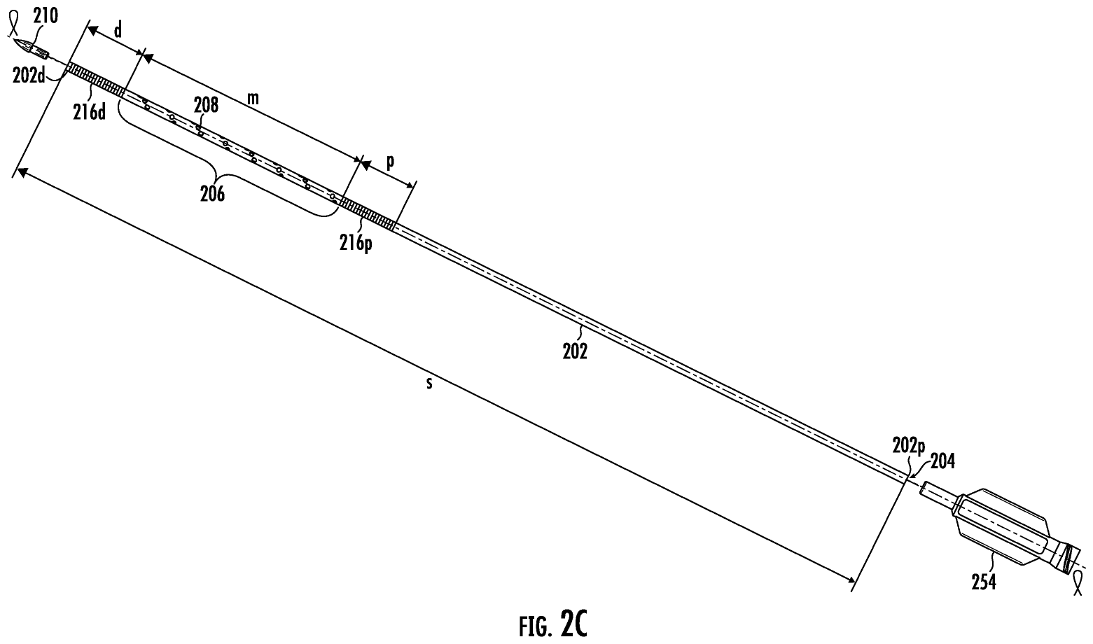

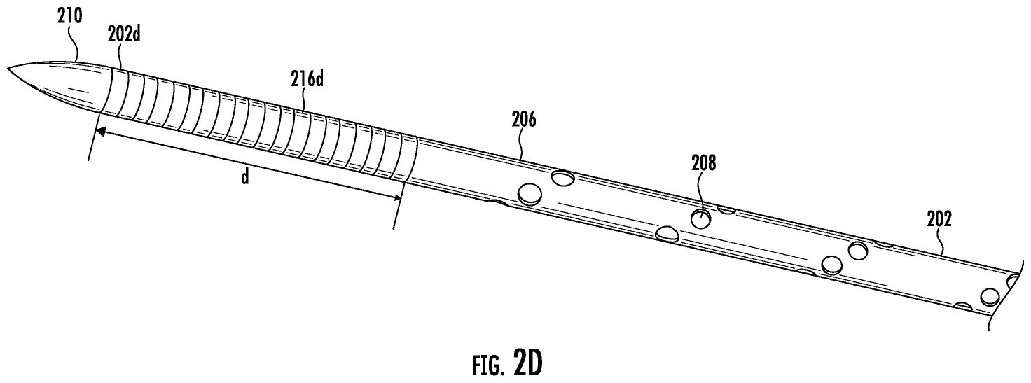

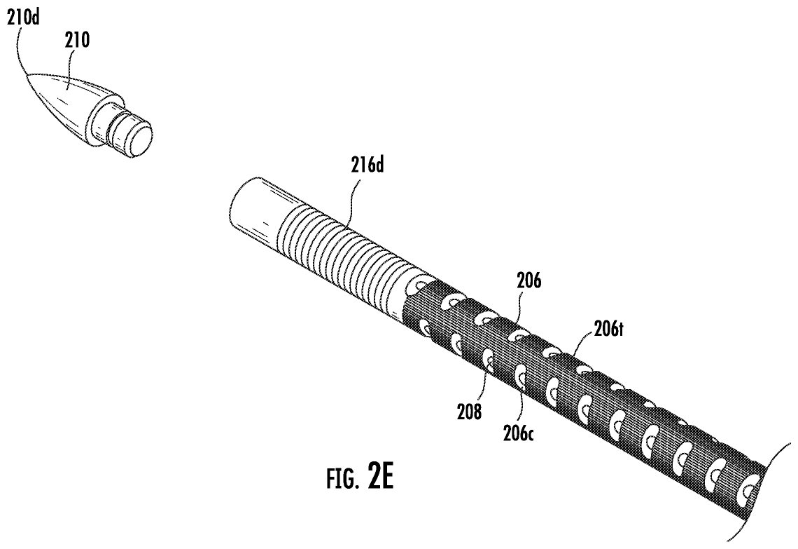

Referring to FIGS. 2A-2D, parts and assembly of a cannula 200 for delivering fluid are illustrated, in accordance with an embodiment of the disclosure. The cannula 200 includes a shaft 202 having a proximal end (not shown), a distal end 202d, and a lumen 204. Radial apertures 208 are disposed along a delivery portion 206 of the shaft 202. The radial apertures 208 are disposed helically about the delivery portion 206 of the shaft 202 and each radial aperture 208 is in fluid communication with the lumen 204.

A body 210 (as best illustrated in FIG. 2B) includes a pointed distal tip 210d and a midportion 210m having a diameter larger than the distal tip 210d. The distal tip 210d gradually widens from a point to the midportion 210 in a proximal direction along a conical convex surface therebetween that may promote a steady increase in peak axial force as the body 210 is axially inserted into tissue substantially along axis α. The pointed distal tip 210d that widens to the midportion 210m may promote dilation of the tissue that the body 210 is inserted into. The body 210 includes a proximal insertion portion 210p having a diameter smaller than the midportion 210m and dimensioned to fit within the lumen 204 of the shaft 202. The diameter of the midportion 210m is substantially equivalent to an outer diameter of the shaft 202, but in various embodiments, the diameter of the midportion 210m may be greater than the diameter of the shaft 202. A transition between the diameters of the midportion 210m to the proximal insertion portion 210p includes a mid bevel 210a. The mid bevel 210a may promote an interference fit and/or seal with the distal end 202d of the shaft 202 with the body 210 inserted within the lumen 204. Additionally or in the alternative, if the midportion 210m has a diameter larger than the outer diameter of the shaft 202, the mid bevel 210a may promote a smooth transition from the larger diameter midportion 210m to the smaller diameter of the shaft 202. The proximal insertion portion 210p includes a proximal bevel 210b at the proximal tip of the proximal insertion portion 210p, which may promote ease of insertion of the body 210 into the lumen 204 of the shaft 202. The body 210 may be secured within the shaft 202 by, e.g., swaging, welding, adhering, interference fit, a combination thereof, or the like. The body 210 may comprise a material different from or the same as that of the shaft 202. For example, in various embodiments, the body 210 may comprise a polymeric material, for example, an epoxy such as EPX 82, available from Carbon, Inc. (Redwood City, Calif., USA), carbon steel, stainless steel, nickel, gold, platinum, an alloy, a combination thereof, or the like. In various embodiments, the body 210 may be formed with or formed from the shaft 202, e.g., the distal end 210d may be formed into a pointed tip such as the portion of the body 210 between the midportion 210m and the distal tip 210d, and the lumen will not extend to the distal end of the shaft.

As best illustrated in FIGS. 2C and 2D, the cannula assembly includes a handle 254, e.g., a luer lock manifold or the like, coupled to the proximal end 202p of the shaft 202 with luminal access to the lumen 204. A deliverable fluid may be introduced through the handle 254, delivered along the lumen 204, and deployed through the apertures 208 along the delivery portion 206. The cannula may be inserted into/across a target tissue by distally leading insertion of the cannula with the body 210 disposed within the lumen 204 at the distal end 202d of the shaft 202. The distal echogenic marker 216d and the proximal echogenic marker 216p may assist with navigating, locating, and/or positioning of the cannula and/or the delivery portion 206 with respect to the target tissue. An alternative embodiment in FIG. 2E illustrates a body 210 having a pointed distal tip 210d, distal echogenic marker 216d, and delivery portion 206, including radial apertures 208. There is expected to be some tissue resistance during side hole injection. To address this effect, the radial apertures 208 open into small reservoirs in the form of concavities 206c in the delivery portion 206. In addition, a series of longitudinal overflow tracks 206t are provided adjacent to the concavities 206c to allow for longitudinal migration of the injected deliverable fluid from the concavities 206c along a length of the delivery portion 206. Such features may be configured to leave a small residue of deliverable fluid upon removal of the needle, for example, to treat the needle track and prevent cancer cell seeding of the track.

In various embodiments, one or more dimensions of features or axial lengths along a longitudinal axis l of the cannula may be variable depending on anatomy, size of target tissue, location of target tissue, target tissue border arrangements, deliverable fluid, type or procedure, etc. The shaft 202 has a length s, e.g., about 18 in. (about 457.2 mm) for accessing a liver, but may be shorter, e.g., for accessing skin or breast tissue. For further examples, a length of a shaft may be less than about 500 mm such as about 5 mm, 10 mm 20 mm, 50 mm, 100 mm, 200 mm, 250 mm, 400 mm, etc. The shaft 202 has a gauge, e.g., about 21.5 gauge (about 0.7 mm). The distal echogenic marker 216d has an axial length d and the proximal echogenic marker 216p has an axial length p, which may be substantially similar to or different from each other, and may be, e.g., about 5 mm. A length m of the delivery portion 206 may be, e.g., about 20 mm, or about 10 mm for a 20 mm target tissue dimension. A diameter of one or more of the apertures 208 may be about 0.010 inches (about 0.254 mm) to about 0.015 inches (about 0.381 mm).

In various embodiments, a shaft may be a laser cut or otherwise machined tube. One or more features of the shaft may also be laser cut or otherwise machined, e.g., apertures, echogenic markers, lumens, pointed tips, etc. A shaft may comprise variable materials depending on parameters of a procedure such as, e.g., an epoxy such as an epoxy such as EPX 82, a polymer, carbon steel, stainless steel, nickel, gold, platinum, an alloy, a metal, a combination thereof, or the like.

In various embodiments, an echogenic marker may include one or more features that assist with visual and/or ultrasonic locating of at least a portion of a device. An echogenic marker may comprise a groove, deformation, etching, carving, or removal of material at a location of a device of interest for tracking during a procedure. An echogenic marker may include a single marker (e.g., a continuous circumferential band) or multiple markers (e.g., multiple continuous bands axially aligned with respect to each other and discontinuous with each other). One or more echogenic markers may be tracked during a procedure such as for delivery, navigating, positioning, locating anatomy, etc.

In various embodiments, radial apertures of a shaft disclosed herein may be disposed along the shaft in various patterns, e.g., arranged as helices, circumferential bands, axial lines, a combination thereof, or the like. Radial apertures of a shaft may be substantially similar to each other or vary. Radial apertures herein may comprise one or more shapes, e.g. circular, oblong, ellipsoidal, rectangular, slotted, a combination thereof, or the like. Some apertures of a shaft may have a smaller dimension and be configured to deploy a fluid while other apertures may have a larger dimension configured to deploy a fluid as well as to elastically and/or plastically deform to assist with navigation and positioning on the shaft during a procedure. In various embodiments, any number of apertures may be used, e.g., 1, 2, 4, 6, 10, 15, 20, 25, 50, 100, etc., or about 20 to about 100 or the like.

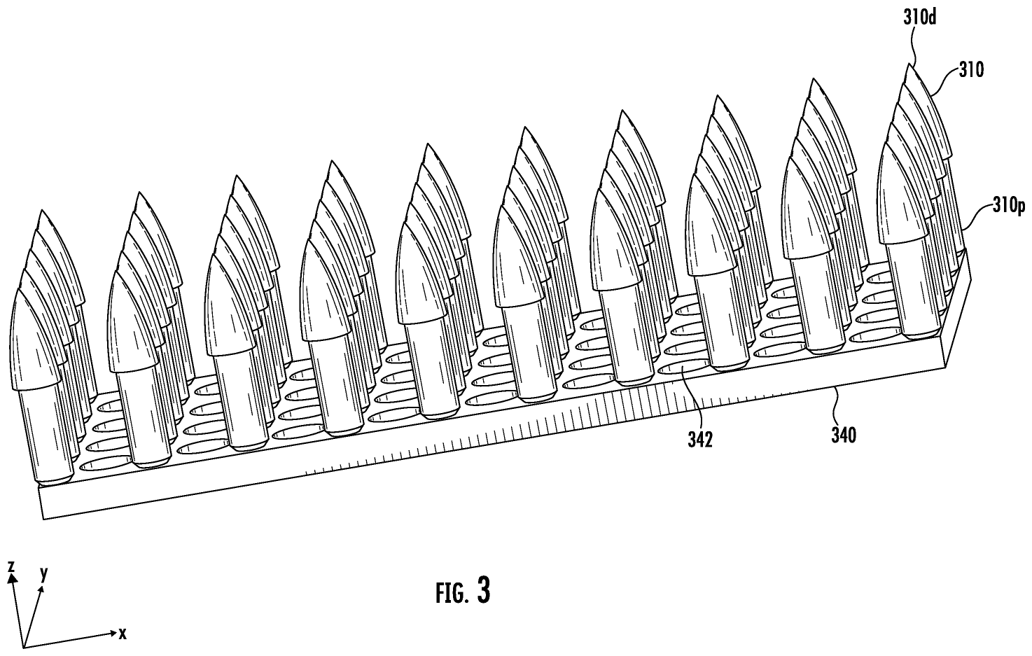

Referring to FIG. 3, a perspective view of an array of bodies 310 is illustrated, according to an embodiment of the disclosure. The bodies 310 are disposed along a stage 340. This arrangement of the stage 340 and array of bodies 310 may be beneficial for manufacturing the bodies 310 in a batch of multiple bodies 310, compared to a single body 310. For example, the array of bodies 310 may be additively manufactured (e.g., three-dimensionally printed, or the like) by establishing the stage 340 at the bottom of the manufactured structure. The stage 340 may include apertures 342 or other voids or thinning of material to decrease material, weight of the structure, cost of the structure, and time to manufacture the structure. The bodies 310 are manufactured on top of the stage 340 and are arrayed in rows and columns along an x-axis and a y-axis, respectively. The bodies 310 are formed from proximal insertion portions 310p on the stage 340 towards distal tips 310d along a z-axis. The bodies 310 may be removed from the stage 340 for assembling into shafts of cannulas described herein. A bevel of a proximal-most portion of the proximal insertion portion 310p of each body 310 may assist with severing each body 310 from the stage 340. Although 50 bodies 310 are illustrated, in various embodiments, any number of bodies 310 on a stage 340 may be formed, e.g., 1, 2, 3, 4, 5, 6, 10, 15, 20, 25, 50, 100, 1000, etc.

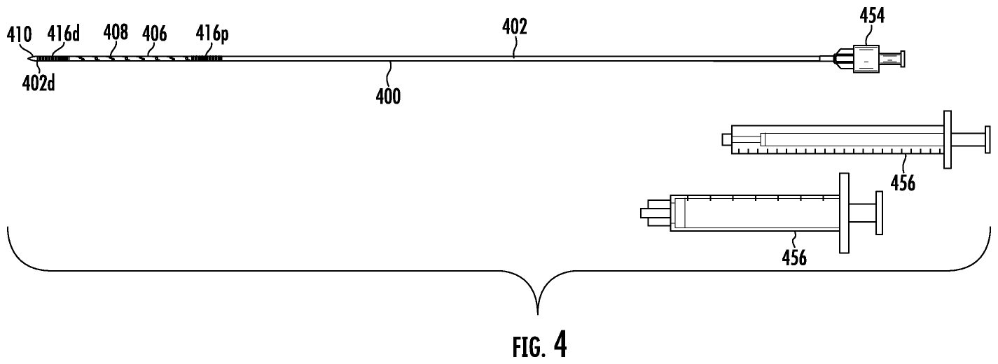

Referring to FIG. 4, a perspective view of a fluid delivery system is illustrated, according to an embodiment of the disclosure. A cannula 400 is illustrated including a proximal handle 454 with luminal access to a lumen of a shaft 402. The shaft 402 includes a plurality of apertures 408 disposed along a delivery portion 406 disposed between a proximal echogenic marker 416p and a distal echogenic marker 416d of the shaft 402. A body 410 having a pointed tip is disposed at a distal end 402d of the shaft 402. The handle 454 is configured to interface with a variety of injectable reservoirs 456 such that a deliverable fluid therein may be delivered from the reservoir 456, through the handle 454, through the shaft 402, and deploy out of the apertures 408.

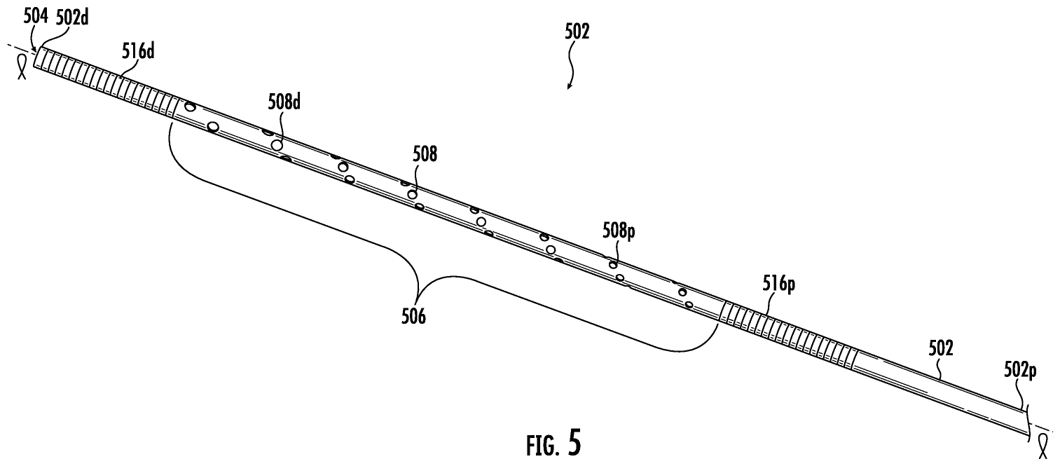

FIG. 5 illustrates a perspective view of a shaft 502, according to an embodiment of the disclosure. The shaft 502 includes a proximal end 502p, a distal end 502d, and a lumen 504. Radial apertures 508 are disposed along a delivery portion 506 of the shaft 502. The delivery portion 506 is disposed between a distal echogenic marker 516d and a proximal echogenic marker 516p along a longitudinal axis l of the shaft 502. The radial apertures 508 are disposed helically about the delivery portion 506 of the shaft 502 and each radial aperture 508 is in fluid communication with the lumen 504. The radial apertures 508 include proximal apertures 508p towards the proximal end 502p having a dimension (e.g., a diameter, a width, etc.) smaller than that of distal apertures 508d towards the distal end 502d. For example, a cross-sectional area (e.g., in a direction normal to flow) of the distal-most aperture may be anywhere from fractions to multiple times larger than a cross-sectional area of the proximal-most aperture. Because the apertures 508 comprises a gradual increase in at least one dimension along the delivery portion 506 from the proximal end 502p towards the distal end 502d, a fluid having a high viscosity (e.g., a viscosity larger than that of water) may more easily deploy out of the larger distal apertures 508d than out of the smaller proximal apertures 508p. Because there is a pressure drop along the length of the shaft 502, such an arrangement may assist with ensuring substantially uniform deployment of fluid out of the apertures 508 along the longitudinal axis l and the deployment portion 506 (i.e., promoting substantially uniform fluid deployment and flowrate along the target tissue as deployment of the fluid may be more restricted at the proximal apertures 508p, where pressure is higher, compared to the distal apertures 508d, where pressure is lower).

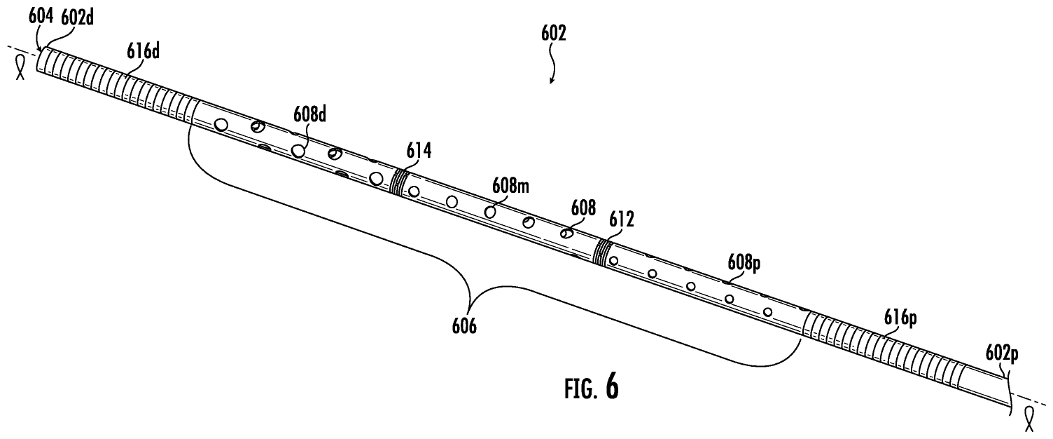

FIG. 6 illustrates a perspective view of a shaft 602, according to an embodiment of the disclosure. The shaft 602 includes a proximal end 602p, a distal end 602d, and a lumen 604. Radial apertures 608 are disposed along a delivery portion 606 of the shaft 602. The delivery portion 606 is disposed between a distal echogenic marker 616d and a proximal echogenic marker 616p along a longitudinal axis l of the shaft 602. The radial apertures 608 are disposed about the delivery portion 606 of the shaft 602 and each radial aperture 608 is in fluid communication with the lumen 604. The radial apertures 608 include proximal apertures 608p towards the proximal end 602p having a dimension (e.g., a diameter, a width, etc.) smaller than that of middle apertures 608m and that of distal apertures 608d towards the distal end 602d. The middle apertures 608m have a dimension larger than that of the proximal apertures 608p, and smaller than that of the distal apertures 608d. Because the apertures 608 comprises a gradual increase in at least one dimension along the delivery portion 606 from the proximal end 602p towards the distal end 602d, a fluid delivered towards the apertures 608 having a high viscosity (e.g., a viscosity larger than that of water) may more easily deploy out of the larger distal apertures 608d than out of the smaller proximal apertures 608p. Because there is a pressure drop along the length of the shaft 602, such an arrangement may assist with ensuring substantially uniform deployment of fluid out of the apertures 608 along the longitudinal axis l and the deployment portion 606 (i.e., promoting substantially uniform fluid deployment and flowrate along the target tissue as deployment of the fluid may be more restricted at the proximal apertures 608p, where pressure is higher, compared to the middle apertures 608m, where the pressure is lower, and the distal apertures 608d where the pressure is even lower). The delivery portion 606 also includes a first flex portion 612 and a second flex portion 614 along its length amongst the apertures 608. The flex portions 612, 614 include circumferential and axial etchings (i.e., grooves) that thin and weaken a wall of the shaft 602 of the delivery portion 606. The flex portions 612, 614 are configured to elastically and/or plastically deform during insertion, steering navigation, and/or positioning of the shaft 602 as desired to maneuver the shaft 602 into a position for optimal delivery of a fluid amongst a target tissue. Although two flex portions 612, 614 are illustrated, in various embodiments any number of flex portions may be utilized, e.g., 0, 1, 3, 4, 5, 6, 8, 10, 15, 20, 50, etc. Various numbers and lengths of flex portions may be utilized to increase or decrease flexibility as desired. Although the flex portions 612, 614 are illustrated with circumferential and axial grooves, a shaft may alternatively include one or more grooves that are solely axial, solely circumferential, solely oblique, solely acute, solely obtuse, and/or a combination thereof In various embodiments, a flex portion may include one or more apertures of various shapes, e.g., circular, oblong, slotted, etc. In various embodiments, it may be desirable for flex portions to minimize curvature of a shaft such that at least some columnar strength of the shaft may be maintained for steering of the device via minute rotational adjustments and distal advances. Repeat adjustments of curvature, advancements, and/or rotation may be made for steering a device towards, within, or around target tissue.

Embodiments of methods of delivering a fluid disclosed herein may include inserting a cannula comprising a shaft, a lumen, and a plurality of radial apertures disposed along a delivery portion of the shaft. Each radial aperture may be in fluid communication with the lumen. At least one echogenic marker may be disposed along the shaft within or adjacent to the delivery portion into a patient. At least one echogenic marker may be detected with ultrasound. The delivery portion may be positioned within a target tissue with the assistance of the at least one echogenic marker. The fluid may be delivered through the lumen and out of the plurality of apertures such that the fluid is delivered to the target tissue. The fluid may comprise a viscosity that varies with temperature, for example the fluid may increase in viscosity as temperature rises and approaches a patient body temperature. The at least one echogenic marker may be positioned at a border of the target tissue. The fluid may be a gellable fluid. The fluid may be maintained at a temperature below about 37° C. at least until delivery. The delivery portion may be oriented along the target tissue by flexing the delivery portion. The fluid may be maintained within the target tissue for a treatment period of at least 24 hours.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A cannula, comprising:
a shaft comprising a proximal end, a distal end, and a lumen;
a plurality of apertures disposed along a delivery portion of the shaft, each aperture in fluid communication with the lumen;
at least one echogenic marker disposed along the shaft within or adjacent to the delivery portion; and
a body disposed within the distal end of the shaft.

2. The cannula of claim 1, wherein the body comprises a pointed distal tip, a midportion having a diameter larger than the distal tip, and a proximal insertion portion having a diameter smaller than the midportion and dimensioned to fit within the distal end of the shaft.

3. The cannula of claim 2, wherein the diameter of the midportion is greater than or equal to a diameter of the shaft.

4. The cannula of claim 1, wherein the cannula comprises from 2 to 100 apertures.

5. The cannula of claim 1, wherein the plurality of apertures radially from a central axis of the shaft.

6. The cannula of claim 1, wherein the plurality of apertures comprises at least one proximal aperture having a smaller width than a width of at least one distal aperture of the plurality of apertures.

7. The cannula of claim 6, wherein the plurality of apertures comprises a gradual increase in width along a length of the shaft between a proximal end of the delivery portion and a distal end of the delivery portion.

8. The cannula of claim 1, wherein a width of at least one of the plurality of apertures is about 0.254 mm±0.100 mm to about 0.381mm±0.100 mm.

9. The cannula of claim 1, wherein an axial length of the delivery portion ranges from about 1.5 cm to about 2.5 cm.

10. The cannula of claim 1, wherein the cannula comprises one or more echogenic markers disposed adjacent a proximal end of the delivery portion and one or more echogenic markers disposed adjacent a distal end of the delivery portion.

11. The cannula of claim 1, wherein the cannula comprises a plurality of echogenic markers in the form of rings disposed adjacent a proximal end of the delivery portion and a plurality of echogenic markers in the form of rings disposed adjacent a distal end of the delivery portion.

12. The cannula of claim 1, wherein an axial length of the at least one echogenic marker ranges from about 2 mm to about 10 mm.

13. A cannula, comprising:
a shaft comprising a proximal end, a distal end, and a lumen therethrough;
a plurality of apertures disposed along a delivery portion of the shaft, each aperture in fluid communication with the lumen;
a distal marker portion longitudinally along the shaft distal to the delivery portion;
a proximal marker portion along the shaft proximal to the delivery portion; and
a body disposed within the distal end of the shaft, the distal body extending distally from the shaft to a gradually pointed tip.

14. The cannula of claim 13, wherein each of the distal marker portion and the proximal marker portion comprises a plurality of echogenic ringed grooves.

15. The cannula of claim 13, wherein the body comprises a pointed distal tip, a midportion having a diameter larger than the distal tip, and a proximal insertion portion having a diameter smaller than the midportion and dimensioned to fit within the distal end of the shaft.

16. The cannula of claim 13, wherein a width of at least one of the plurality of apertures is about 0.254 mm to about 0.381 mm.

17. A method of delivering a fluid comprising:
inserting a cannula comprising a shaft, a lumen, a plurality of apertures disposed along a delivery portion of the shaft, each aperture in fluid communication with the lumen, and at least one echogenic marker disposed along the shaft within or adjacent to the delivery portion;
detecting the at least one echogenic marker with ultrasound;
positioning the delivery portion within a target tissue with the assistance of the at least one echogenic marker; and
delivering the fluid through the lumen and out of the plurality of apertures such that the fluid is delivered to the target tissue.

18. The method of claim 17, wherein the fluid comprises a viscosity that increases as the fluid approaches a body temperature of a patient.

19. The method of claim 17, further comprising positioning the at least one echogenic marker at a border of the target tissue.

20. The method of claim 17, wherein delivering the fluid further comprises maintaining the fluid within the target tissue for a treatment period of at least 24 hours.
